(19) United States
(12) Patent Application Publication
(10) Pub. No.: US 2018/0310644 A1
(43) Pub. Date: Nov. 1, 2018

(54) Connector Integration for Smart Clothing

(71) Applicant: Google LLC, Mountain View, CA (US)

(72) Inventors: Ivan Poupyrev, Sunnyvale, CA (US); Leonardo Giusti, Mountain View, CA (US); Mauricio Gutierrez Bravo, Mountain View, CA (US)

(21) Appl. No.: 15/963,552

(22) Filed: Apr. 26, 2018

Related U.S. Application Data
(60) Provisional application No. 62/491,015, filed on Apr. 27, 2017.

(51) Int. Cl. A41D 1/00; G06F 3/044; G08C 17/02; D02G 3/44; A41D 1/04; A43B 3/00; D04B 1/24; A41B 1/08; A63B 43/00; A63B 69/00; A63B 71/06; D04D 9/00; D04B 21/20; A45C 3/06; A61B 5/0408; A61B 5/0478; A61B 5/00

(52) U.S. Cl. CPC A41D 1/005; D10B 2401/16; G08C 17/02; D02G 3/441; A41D 1/04; A43B 3/0005; D04B 1/246; A41B 1/08; A63B 43/004; A63B 69/002; A63B 71/0622; D04D 9/00; D04B 21/207; A45C 3/06; A61B 5/04085; A61B 5/0478; A61B 5/6897; A61B 5/6804; G08C 2201/32; G06F 2203/04102; A41D 2200/20; D10B 2501/00; A63B 2225/50; A63B 2243/0025; D10B 2501/043; A63B 2220/17; A63B 2071/0655; G06F 3/044

(57) ABSTRACT

This document describes an interactive object with at least one electronics module and a touch sensor. The interactive object may be a garment, garment accessory, or garment container. The interactive object may be configured to provide at least a haptic, audio, or visual output. The interactive object may also contain conductive threads and a touch sensor containing said conductive threads.

### PRIORITY CLAIM

This application is based upon and claims priority to U.S. Patent Application Ser. No. 62/491,015, filed Apr. 27, 2017, and entitled “CONNECTOR INTEGRATION FOR SMART CLOTHING,” the disclosure of which is incorporated by reference herein in its entirety.

### BACKGROUND

An interactive textile includes conductive thread woven into the interactive textile to form a capacitive touch sensor that is configured to detect touch-input. The interactive textile can process the touch-input to generate touch data that is useable to initiate functionality at various remote devices that are wirelessly coupled to the interactive textile and/or to objects incorporating the interactive textile. For example, the interactive textile may aid users in controlling volume on a stereo, pausing a movie playing on a television, or selecting a webpage on a desktop computer. Due to the flexibility of textiles, the interactive textile may be easily integrated within flexible or hard objects, such as clothing, handbags, fabric casings, hats, and so forth.

The interactive textile includes a grid or array of conductive thread woven into the interactive textile. Each conductive thread includes a conductive wire (e.g., a copper wire) that is twisted, braided, or wrapped with one or more flexible threads (e.g., polyester or cotton threads). Although interactive textiles have provided great advancements in the art, further improvements are needed. For example, greater responsiveness and/or controls are needed for further advancing the art.

### SUMMARY

This document describes techniques and apparatuses for connecting an electronic component to an interactive textile. An interactive textile may include electronic components including sensors, such as conductive thread woven into the interactive textile to form a capacitive touch sensor that is configured to detect touch-input.

This document also describes an interactive object with multiple electronic modules. An internal electronics module includes a first subset of electronic components, such as sensing circuitry configured to detect and receive user-inputted actions. The interactive object may also interact with a controller configured to receive information from the internal electronics module based upon the user-inputted actions. An output device may be in communication with the controller. Based on input from the controller, the output device can provide at least a haptic response, an audio response, or a visual response.

In some embodiments, the interactive object may be a garment (e.g. clothes, athletic clothes, outerwear, hooded garment), garment accessory (e.g. belt, headwear), or garment container (e.g. handbag, duffel bag, purse, backpack).

In some embodiments, the interactive object may produce a haptic response. For example, a user-interactive garment, may contain a compression device, such as contractible threads or bands. For example, the compression device may expand or contract at least a portion of the garment. The expansion or contraction may operate responsive to a user-inputted action on a touch sensor. For example, athletic pants and an athletic bra may be outfitted accordingly.

In some embodiments, the interactive object may be an article of clothing. The article of clothing may contain an internal electronics module containing a sensor. The motion of an article of clothing by a wearer is detected and communicated to a controller. In some embodiments, action or motion input from the user is used to record exercise motions performed by the user. The controller is configured to track movements of the wearer and cause a display of information related to the movements on a display device. In another embodiment, a shoe may be modified similarly. A garment configured according to this embodiment may track the movements of the wearer during an athletic performance (e.g. a basketball player, a professional football player). The data so obtained may be compared to data from a reference performance obtained from another wearer. For example, a wearer may compare a recent personal performance to a pre-programmed performance of a professional athlete.

In another embodiment, the interactive article of clothing contains touch or motion sensors. For example, a shirt with opposing sleeves can contain a display device located on one of the shirt sleeves. The sensors may relay data to a controller which may cause the display of information related to the motion of the wearer on the display device (e.g. the speed of the wearer, a distance the wearer has moved, a distance remaining to a destination). Other sensors might provide additional information: e.g. an amount of time the wearer has been moving, or one or more vital signs of the wearer. Such information may be explicit (e.g. textual) or may be symbolic (e.g. the intensity of a glowing light mapped to distance the wearer has traveled). In addition or in alternative to measured information, garments configured with or without sensors may contain light emitting diodes controlled to illuminate according to a pattern that corresponds to a beat of an audio track that the wearer is enjoying.

In another embodiment, the interactive object may be a garment container, such as a handbag. An internal electronics module may contain a global positioning device (GPS) which can track the location of the garment container over time. An output device may be configured to display the tracked locations where the garment container has traveled. Optionally, the display device may display the locations along a timeline. In some embodiments, the garment container communicates with a controller, such as a mobile device. If the mobile device has a camera, the controller may be configured to coordinate pictures taken with the camera to locations traveled by the garment container.

In another example, a hooded garment incorporates an interactive textile as a drawstring in communication with an internal electronics module and a controller. An output device provides an audio response (e.g. music) to user-inputted actions on the drawstring, the audio response projected through at least one speaker, which may optionally be connected to the hood of the hooded garment. The hooded garment may include a network interface over which the user-inputted actions may be communicated to the controller (e.g. a mobile device, such as a smartphone).

This summary is provided to introduce simplified concepts concerning an interactive object with multiple electronics modules, which is further described below in the Detailed Description. This summary is not intended to identify essential features of the claimed subject matter, nor is it intended for use in determining the scope of the claimed subject matter.

### BRIEF DESCRIPTION OF THE DRAWINGS

Embodiments of an interactive object with multiple electronics modules are described with reference to the following drawings. The same numbers are used throughout the drawings to reference like features and components:

FIG. 1 is an illustration of an example environment in which an interactive textile with multiple electronics modules can be implemented.

FIG. 2 illustrates an example system that includes an interactive object and multiple electronics modules.

FIG. 3 illustrates an example of an interactive object with multiple electronics modules in accordance with one or more implementations.

FIG. 4 illustrates an example of a connector for connecting an external communications module to an interactive object in accordance with one or more implementations.

FIG. 5 illustrates an example of an internal electronics module shown connected to a plurality of conductive threads.

FIG. 6 illustrates an external electronics module in accordance with one or more implementations.

FIG. 7 illustrates various components of an example computing system that can be implemented as any type of client, server, and/or computing device as described with reference to the previous FIGS. 1-6 to implement an interactive object with multiple electronics modules.

FIG. 8 illustrates an example of a fabric made in accordance with the present disclosure including conductive yarns.

FIG. 9 illustrates an example of a fabric made in accordance with the present disclosure including conductive yarns.

FIG. 10 illustrates an example of an elastic fabric made in accordance with one or more implementations including conductive yarns.

FIG. 11 illustrates one embodiment of an electronics module in accordance with the present disclosure.

FIG. 12 illustrates an example of a flexible haptics device made in accordance with the present disclosure.

FIG. 13 illustrates one embodiment of an interactive garment made in accordance with the present disclosure.

FIG. 14 illustrates a portion of the interactive garment illustrated in FIG. 13.

FIG. 15 illustrates examples of electrically conductive ribbons and a network made from the ribbons in accordance with the present disclosure.

FIG. 16 illustrates a fabric made in accordance with the present disclosure embedded with light-emitting diodes.

FIG. 17 illustrates one embodiment of an electronics module made in accordance with the present disclosure connected to a flexible organic light-emitting diode.

FIG. 18 illustrates a touch cord made in accordance with the present disclosure.

FIG. 19 illustrates various illuminated yarns or threads made in accordance with the present disclosure.

FIG. 20 illustrates one embodiment of an interactive garment made in accordance with the present disclosure.

FIG. 21 illustrates another embodiment of an interactive garment made in accordance with the present disclosure.

FIG. 22 illustrates a portion of the interactive garment illustrated in FIG. 21.

FIG. 23 illustrates yet another embodiment of an interactive garment made in accordance with the present disclosure.

FIG. 24 illustrates still another embodiment of an interactive garment made in accordance with the present disclosure.

FIG. 25 illustrates another embodiment of an interactive garment made in accordance with the present disclosure.

FIG. 26 illustrates further embodiments of interactive garments made in accordance with the present disclosure.

FIG. 27 illustrates yet another embodiment of an interactive garment made in accordance with the present disclosure.

FIG. 28 illustrates an interactive athletic ball made in accordance with the present disclosure.

FIG. 29 illustrates yet another embodiment of an interactive garment made in accordance with the present disclosure.

FIG. 30 illustrates another view of the interactive garment illustrated in FIG. 27.

FIG. 31 illustrates one embodiment of an interactive handbag or luggage made in accordance with the present disclosure.

FIG. 32 illustrates yet another embodiment of an interactive garment made in accordance with the present disclosure.

### DETAILED DESCRIPTION

Overview

Electronics embedded in garments and other flexible objects (e.g., blankets, handbags, and hats) are becoming increasingly common. Such electronics often need connectivity to external devices for power and/or data transmission. For example, it can be difficult to integrate bulky electronic components (e.g., batteries, microprocessors, wireless units, and sensors) into wearable garments, such as a shirt, coat, a shoe, or pair of pants. Furthermore, connecting such electronic components to a garment may cause issues with durability since garments are often washed. However, some electronic components, such as sensing circuity, are better equipped to be positioned within the garment.

An interactive object that may include multiple electronics modules is described. An interactive object (e.g., a garment) includes at least an internal electronics module containing a first subset of electronic components for the interactive object, and optionally an external electronics module containing a second subset of electronic components for the interactive object. As described herein, the internal electronics module may be physically and permanently coupled to the interactive object, whereas the external electronics module may be removably coupled to the interactive object. Thus, instead of integrating all of the electronics within the interactive object, at least some of the electronics are placed in the external electronics module.

In one or more implementations, the interactive object includes an interactive textile with conductive threads woven into the textile to form a flexible touch pad. The internal electronics module contains sensing circuity that is directly coupled to the conductive threads to enable the detection of touch-input to the interactive textile. The external electronics module contains electronic components that are needed to process and communicate the touch-input data, such as a microprocessor, a power source, a network interface, and so forth.

The interactive object further includes a communication interface configured to enable communication between the internal electronics module and an external electronics module or other device, such as a mobile phone. In some implementations, the communication interface may be implemented as a connector that connects the electronic components in the external electronics module to the electronic components in the internal electronics module to enable the transfer of power and data between the modules. The connector may include a connector plug and a connector receptacle. For example, the connector plug may be implemented at the external electronics module and is configured to connect to the connector receptacle, which may be implemented at the interactive object.

Thus, while the electronic components can be separated into multiple different modules, the communication interface enables the system to function as a single unit. For example, the power source contained within the external electronics module may transfer power, via the communication interface, to the sensing circuity of the internal electronics module to enable the sensing circuitry to detect touch-input to the conductive thread. When touch-input is detected by the sensing circuity of the internal electronics module, data representative of the touch-input may be communicated, via the communication interface, to the microprocessor contained within the external electronics module. The microprocessor may then analyze the touch-input data to generate one or more control signals, which may then be communicated to a remote computing device (e.g., a smart phone) via the network interface to cause the computing device to initiate a particular functionality.

Separating the electronics of the interactive object into multiple different modules provides a variety of different benefits. For example, the system design enables interoperability and customization because the external electronics module can be detached from the interactive object, and then attached to a different interactive object to carry over some of the functions and properties, such as user specific settings. Additionally, by separating the garment embedded electronics from the external electronics module, users, designers and companies are able to design the external electronics modules in the form factor, mechanical, material and surface finish qualities that are specific to the application or the user. For example, a leather jacket might have an external electronics module that is leather, and in the form of a strap that matches a certain jacket style, or allows a flexible form factor that would have been hard to achieve inside a garment.

Furthermore, separating the electronics enable broken parts to be easily replaced or serviced without the need to access the entire interactive object. For example, the external electronics module can be shipped to a repair service, or a new external electronics module can be purchased without the need to purchase a new interactive object. In addition, separating the electronic components into internal and external modules ensures that parts such as batteries are not exposed to washing cycles that a typical garment would go through. For example, the external electronics module, which may include the battery, can easily be removed from the interactive object before washing the interactive object. Furthermore, by separating parts, the manufacturing challenges are significantly simplified and certification processes (such as FCC certification for RF transmission units) can be handled over the part in question, thereby reducing the complexity.

Example Environment

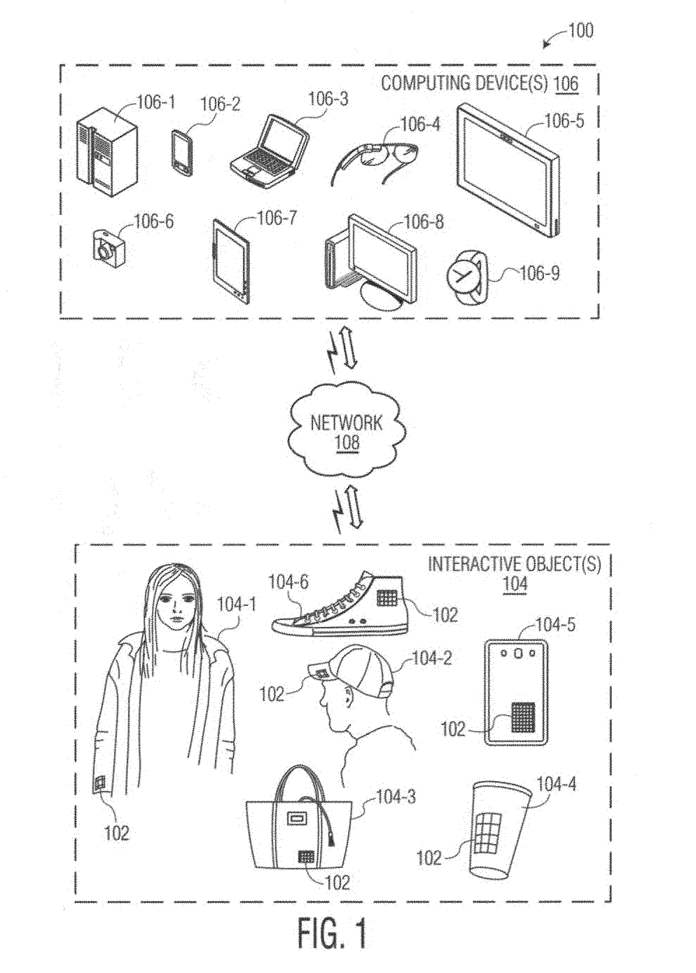

FIG. 1 is an illustration of an example environment 100 in which an interactive textile with multiple electronics modules can be implemented. Environment 100 includes an interactive textile 102, which is shown as being integrated within various interactive objects 104. Interactive textile 102 is a textile that is configured to sense multi-touch-input. As described herein, a textile corresponds to any type of flexible woven material consisting of a network of natural or artificial fibers, often referred to as thread or yarn. Textiles may be formed by weaving, knitting, crocheting, knotting, pressing threads together or consolidating fibers or filaments together in a nonwoven manner.

In environment 100, interactive objects 104 include “flexible” objects, such as a shirt 104-1, a hat 104-2, a handbag 104-3 and a shoe 104-6. It is to be noted, however, that interactive textile 102 may be integrated within any type of flexible object made from fabric or a similar flexible material, such as garments or articles of clothing, garment accessories, garment containers, blankets, shower curtains, towels, sheets, bed spreads, or fabric casings of furniture, to name just a few. Examples of garment accessories may include sweat-wicking elastic bands to be worn around the head, wrist, or bicep. Other examples of garment accessories may be found in various wrist, arm, shoulder, knee, leg, and hip braces or compression sleeves. Headwear is another example of a garment accessory, e.g. sun visors, caps, and thermal balaclavas. Examples of garment containers may include waist or hip pouches, backpacks, handbags, satchels, hanging garment bags, and totes. Garment containers may be worn or carried by a user, as in the case of a backpack, or may hold their own weight, as in rolling luggage. Interactive textile 102 may be integrated within flexible objects 104 in a variety of different ways, including weaving, sewing, gluing, and so forth.

In this example, objects 104 further include “hard” objects, such as a plastic cup 104-4 and a hard smart phone casing 104-5. It is to be noted, however, that hard objects 104 may include any type of “hard” or “rigid” object made from non-flexible or semi-flexible materials, such as plastic, metal, aluminum, and so on. For example, hard objects 104 may also include plastic chairs, water bottles, plastic balls, or car parts, to name just a few. In another example, hard objects 104 may also include garment accessories such as chest plates, helmets, goggles, shin guards, and elbow guards. Alternatively, the hard or semi-flexible garment accessory may be embodied by a shoe, cleat, boot, or sandal. Interactive textile 102 may be integrated within hard objects 104 using a variety of different manufacturing processes. In one or more implementations, injection molding is used to integrate interactive textiles 102 into hard objects 104.

Interactive textile 102 enables a user to control object 104 that the interactive textile 102 is integrated with, or to control a variety of other computing devices 106 via a network 108. Computing devices 106 are illustrated with various non-limiting example devices: server 106-1, smart phone 106-2, laptop 106-3, computing spectacles 106-4, television 106-5, camera 106-6, tablet 106-7, desktop 106-8, and smart watch 106-9, though other devices may also be used, such as home automation and control systems, sound or entertainment systems, home appliances, security systems, netbooks, and e-readers. Note that computing device 106 can be wearable (e.g., computing spectacles and smart watches), non-wearable but mobile (e.g., laptops and tablets), or relatively immobile (e.g., desktops and servers).

Network 108 includes one or more of many types of wireless or partly wireless communication networks, such as a local-area-network (LAN), a wireless local-area-network (WLAN), a personal-area-network (PAN), a wide-area-network (WAN), an intranet, the Internet, a peer-to-peer network, point-to-point network, a mesh network, and so forth.

Interactive textile 102 can interact with computing devices 106 by transmitting touch data through network 108. Computing device 106 uses the touch data to control computing device 106 or applications at computing device 106. As an example, consider that interactive textile 102 integrated at shirt 104-1 may be configured to control the user's smart phone 106-2 in the user's pocket, television 106-5 in the user's home, smart watch 106-9 on the user's wrist, or various other appliances in the user's house, such as thermostats, lights, music, and so forth. For example, the user may be able to swipe up or down on interactive textile 102 integrated within the user's shirt 104-1 to cause the volume on television 106-5 to go up or down, to cause the temperature controlled by a thermostat in the user's house to increase or decrease, or to turn on and off lights in the user's house. Note that any type of touch, tap, swipe, hold, or stroke gesture may be recognized by interactive textile 102.

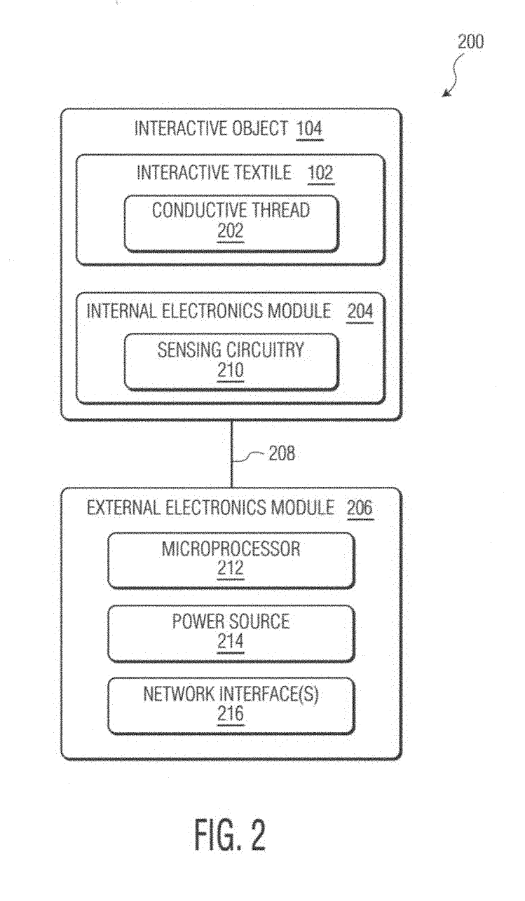

In more detail, consider FIG. 2 which illustrates an example system 200 that includes an interactive object and multiple electronics modules. In system 200, interactive textile 102 is integrated in an object 104, which may be implemented as a flexible object (e.g., shirt 104-1, hat 104-2, or handbag 104-3) or a hard object (e.g., plastic cup 104-4 or smart phone casing 104-5).

Interactive textile 102 is configured to sense multi-touch-input from a user when one or more fingers of the user's hand touch interactive textile 102. Interactive textile 102 may also be configured to sense full-hand touch-input from a user, such as when an entire hand of the user touches or swipes interactive textile 102. To enable the detection of touch-input, interactive textile 102 includes conductive threads 202, which are woven into interactive textile 102 (e.g., in a grid, array or parallel pattern). Notably, the conductive threads 202 do not alter the flexibility of interactive textile 102, which enables interactive textile 102 to be easily integrated within interactive objects 104.

Interactive object 104 includes an internal electronics module 204 that is embedded within interactive object 104 and is directly coupled to conductive threads 202. Internal electronics module 204 can be communicatively coupled to an external electronics module 206 via a communication interface 208. Internal electronics module 204 contains a first subset of electronic components for the interactive object 104, and external electronics module 206 contains a second, different, subset of electronics components for the interactive object 104. As described herein, the internal electronics module 204 may be physically and permanently embedded within interactive object 104, whereas the external electronics module 206 may be removably coupled to interactive object 104.

In system 200, the electronic components contained within the internal electronics module 204 includes sensing circuity 210 that is coupled to conductive thread 202 that is woven into interactive textile 102. For example, wires from the conductive threads 202 may be connected to sensing circuitry 210 using flexible PCB, creping, gluing with conductive glue, soldering, and so forth. In one embodiment, the sensing circuitry 210 can be configured to detect a user-inputted touch-input on the conductive threads that is pre-programmed to indicate a certain request. In one embodiment, when the conductive threads form a grid or other pattern, sensing circuitry 210 can be configured to also detect the location of the touch-input on conductive thread 202, as well as motion of the touch-input. For example, when an object, such as a user's finger, touches conductive thread 202, the position of the touch can be determined by sensing circuitry 210 by detecting a change in capacitance on the grid or array of conductive thread 202. The touch-input may then be used to generate touch data usable to control computing device 106. For example, the touch-input can be used to determine various gestures, such as single-finger touches (e.g., touches, taps, and holds), multi-finger touches (e.g., two-finger touches, two-finger taps, two-finger holds, and pinches), single-finger and multi-finger swipes (e.g., swipe up, swipe down, swipe left, swipe right), and full-hand interactions (e.g., touching the textile with a user's entire hand, covering textile with the user's entire hand, pressing the textile with the user's entire hand, palm touches, and rolling, twisting, or rotating the user's hand while touching the textile).

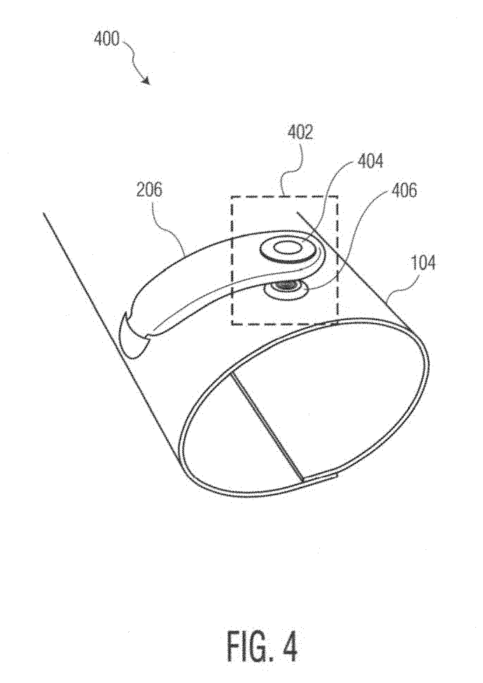

Communication interface 208 enables the transfer of power and data (e.g., the touch-input detected by sensing circuity 210) between the internal electronics module 204 and the external electronics module 206. In some implementations, communication interface 208 may be implemented as a connector that includes a connector plug and a connector receptacle. The connector plug may be implemented at the external electronics module 206 and is configured to connect to the connector receptacle, which may be implemented at the interactive object 104. A more-detailed discussion of example connectors is discussed below with regards to FIGS. 4 and 11-12.

In system 200, the external electronics module 206 includes a microprocessor 212, power source 214, and network interface 216. Power source 214 may be coupled, via communication interface 208, to sensing circuitry 210 to provide power to sensing circuitry 210 to enable the detection of touch-input, and may be implemented as a small battery. When touch-input is detected by sensing circuity 210 of the internal electronics module 204, data representative of the touch-input may be communicated, via communication interface 208, to microprocessor 212 of the external electronics module 206. Microprocessor 212 may then analyze the touch-input data to generate one or more control signals, which may then be communicated to computing device 106 (e.g., a smart phone) via the network interface 216 to cause the computing device 106 to initiate a particular functionality. Generally, network interfaces 216 are configured to communicate data, such as touch data, over wired, wireless, or optical networks to computing devices 106. By way of example and not limitation, network interfaces 216 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like (e.g., through network 108 of FIG. 1).

While internal electronics module 204 and external electronics module 206 are illustrated and described as including specific electronic components, it is to be appreciated that these modules may be configured in a variety of different ways. For example, in some cases, electronic components described as being contained within internal electronics module 204 may be at least partially implemented at the external electronics module 206, and vice versa. Furthermore, internal electronics module 204 and external electronics module 206 may include electronic components other that those illustrated in FIG. 2, such as sensors, light sources (e.g., LED's), displays, speakers, and so forth.

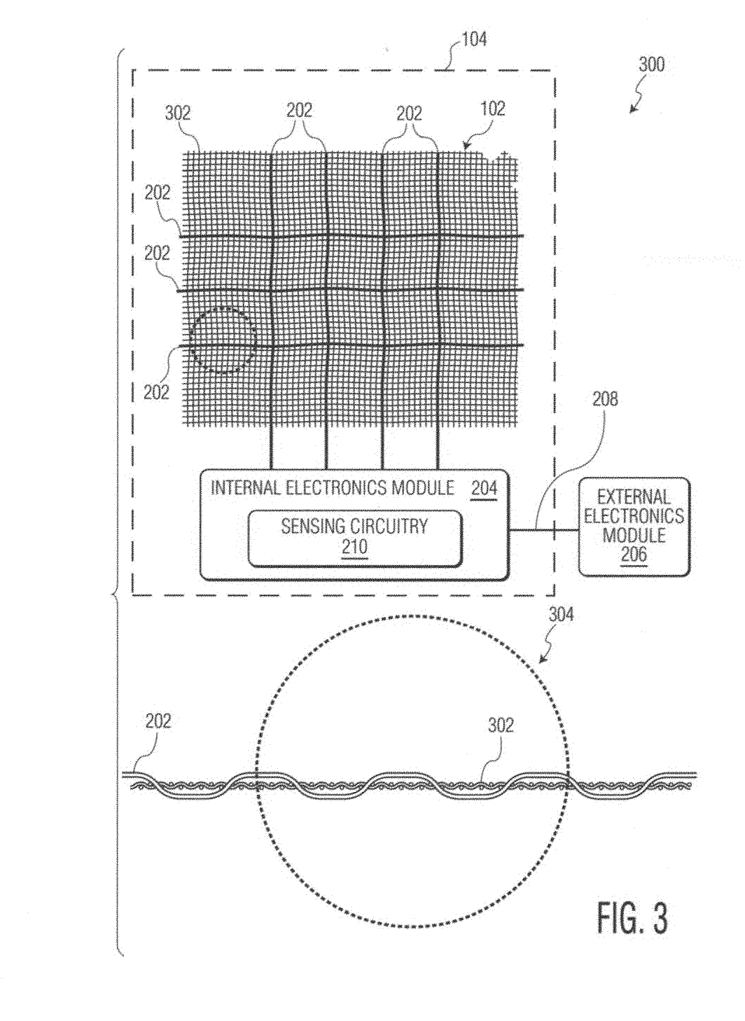

FIG. 3 illustrates an example 300 of interactive object 104 with multiple electronics modules in accordance with one or more implementations. In this example, interactive textile 102 of the interactive object 104 includes non-conductive threads 302 woven with conductive threads 202 to form interactive textile 102. Non-conductive threads 302 may correspond to any type of non-conductive thread, fiber, or fabric, such as cotton, wool, silk, nylon, polyester, and so forth.

At 304, a zoomed-in view of conductive thread 202 is illustrated. Conductive thread 202 includes a conductive wire or a plurality of conductive filaments that are twisted, braided, or wrapped with a flexible thread. As shown, the conductive thread 202 can be woven with an integrated with the non-conductive threads 302 to form a fabric or a textile.

In one or more implementations, conductive thread 202 includes a thin copper wire. It is to be noted, however, that the conductive thread 202 may also be implemented using other materials, such as silver, gold, or other materials coated with a conductive polymer. The conductive thread 202 may include an outer cover layer formed by braiding together non-conductive threads. The non-conductive threads may be implemented as any type of flexible thread or fiber, such as cotton, wool, silk, nylon, polyester, and so forth.

Interactive textile 102 can be formed cheaply and efficiently, using any conventional weaving process (e.g., jacquard weaving or 3D-weaving), which involves interlacing a set of longer threads (called the warp) with a set of crossing threads (called the weft). Weaving may be implemented on a frame or machine known as a loom, of which there are a number of types. Thus, a loom can weave non-conductive threads 302 with conductive threads 202 to create interactive textile 102.

The conductive threads 202 can be woven into the textile 102 in any suitable pattern or array. In one embodiment, for instance, the conductive threads 202 may form a single series of parallel threads. For instance, in one embodiment, the capacitive touch sensor may comprise a single plurality of parallel conductive threads conveniently located on the interactive object, such as on the sleeve of a jacket.

In an alternative embodiment, the conductive threads 202 may form a grid as shown in FIG. 3.

In example 300, conductive thread 202 is woven into interactive textile 102 to form a grid that includes a set of substantially parallel conductive threads 202 and a second set of substantially parallel conductive threads 202 that crosses the first set of conductive threads to form the grid. In this example, the first set of conductive threads 202 are oriented horizontally and the second set of conductive threads 202 are oriented vertically, such that the first set of conductive threads 202 are positioned substantially orthogonal to the second set of conductive threads 202. It is to be appreciated, however, that conductive threads 202 may be oriented such that crossing conductive threads 202 are not orthogonal to each other. For example, in some cases crossing conductive threads 202 may form a diamond-shaped grid. While conductive threads 202 are illustrated as being spaced out from each other in FIG. 3, it is to be noted that conductive threads 202 may be weaved very closely together. For example, in some cases two or three conductive threads may be weaved closely together in each direction. Further, in some cases the conductive threads may be oriented as parallel sensing lines that do not cross or intersect with each other.

In example 300, sensing circuity 210 is shown as being integrated within object 104, and is directly connected to conductive threads 202. During operation, sensing circuitry 210 can determine positions of touch-input on the grid of conductive thread 202 using self-capacitance sensing or projective capacitive sensing.

For example, when configured as a self-capacitance sensor, sensing circuitry 210 charges crossing conductive threads 202 (e.g., horizontal and vertical conductive threads) by applying a control signal (e.g., a sine signal) to each conductive thread 202. When an object, such as the user's finger, touches the grid of conductive thread 202, the conductive threads 202 that are touched are grounded, which changes the capacitance (e.g., increases or decreases the capacitance) on the touched conductive threads 202.

Sensing circuitry 210 uses the change in capacitance to identify the presence of the object. To do so, sensing circuitry 210 detects a position of the touch-input by detecting which horizontal conductive thread 202 is touched, and which vertical conductive thread 202 is touched by detecting changes in capacitance of each respective conductive thread 202. Sensing circuitry 210 uses the intersection of the crossing conductive threads 202 that are touched to determine the position of the touch-input on the grid of conductive threads 202. For example, sensing circuitry 210 can determine touch data by determining the position of each touch as X,Y coordinates on the grid of conductive thread 202.

When implemented as a self-capacitance sensor, “ghosting” may occur when multi-touch-input is received. Consider, for example, that a user touches the grid of conductive thread 202 with two fingers. When this occurs, sensing circuitry 210 determines X and Y coordinates for each of the two touches. However, sensing circuitry 210 may be unable to determine how to match each X coordinate to its corresponding Y coordinate. For example, if a first touch has the coordinates X1, Y1 and a second touch has the coordinates X4,Y4, sensing circuitry 210 may also detect “ghost” coordinates X1, Y4 and X4,Y1.

In one or more implementations, sensing circuitry 210 is configured to detect “areas” of touch-input corresponding to two or more touch-input points on the grid of conductive thread 202. Conductive threads 202 may be weaved closely together such that when an object touches the grid of conductive thread 202, the capacitance will be changed for multiple horizontal conductive threads 202 and/or multiple vertical conductive threads 202. For example, a single touch with a single finger may generate the coordinates X1,Y1 and X2,Y1. Thus, sensing circuitry 210 may be configured to detect touch-input if the capacitance is changed for multiple horizontal conductive threads 202 and/or multiple vertical conductive threads 202. Note that this removes the effect of ghosting because sensing circuitry 210 will not detect touch-input if two single-point touches are detected which are spaced apart.

Alternately, when implemented as a projective capacitance sensor, sensing circuitry 210 charges a single set of conductive threads 202 (e.g., horizontal conductive threads 202) by applying a control signal (e.g., a sine signal) to the single set of conductive threads 202. Then, sensing circuitry 210 senses changes in capacitance in the other set of conductive threads 202 (e.g., vertical conductive threads 202).

In this implementation, vertical conductive threads 202 are not charged and thus act as a virtual ground. However, when horizontal conductive threads 202 are charged, the horizontal conductive threads capacitively couple to vertical conductive threads 202. Thus, when an object, such as the user's finger, touches the grid of conductive thread 202, the capacitance changes on the vertical conductive threads (e.g., increases or decreases). Sensing circuitry 210 uses the change in capacitance on vertical conductive threads 202 to identify the presence of the object. To do so, sensing circuitry 210 detects a position of the touch-input by scanning vertical conductive threads 202 to detect changes in capacitance. Sensing circuitry 210 determines the position of the touch-input as the intersection point between the vertical conductive thread 202 with the changed capacitance, and the horizontal conductive thread 202 on which the control signal was transmitted. For example, sensing circuitry 210 can determine touch data by determining the position of each touch as X,Y coordinates on the grid of conductive thread 202.

Whether implemented as a self-capacitance sensor or a projective capacitance sensor, the conductive thread 202 and sensing circuitry 210 is configured to communicate the touch data that is representative of the detected touch-input to external electronics module 206, which is removably coupled to interactive object 104 via communication interface 208. The microprocessor 212 may then cause communication of the touch data, via network interface 216, to computing device 106 to enable the device to determine gestures based on the touch data, which can be used to control object 104, computing device 106, or applications implemented at computing device 106.

The computing device 106 can be implemented to recognize a variety of different types of gestures, such as touches, taps, swipes, holds, and covers made to interactive textile 102. To recognize the various different types of gestures, the computing device can be configured to determine a duration of the touch, swipe, or hold (e.g., one second or two seconds), a number of the touches, swipes, or holds (e.g., a single tap, a double tap, or a triple tap), a number of fingers of the touch, swipe, or hold (e.g., a one finger-touch or swipe, a two-finger touch or swipe, or a three-finger touch or swipe), a frequency of the touch, and a dynamic direction of a touch or swipe (e.g., up, down, left, right). With regards to holds, the computing device 106 can also determine an area of the grid of conductive thread 202 that is being held (e.g., top, bottom, left, right, or top and bottom. Thus, the computing device 106 can recognize a variety of different types of holds, such as a cover, a cover and hold, a five finger hold, a five finger cover and hold, a three finger pinch and hold, and so forth.

In one or more implementations, communication interface 208 is implemented as a connector that is configured to connect external electronics module 206 to internal electronics module 204 of interactive object 104. Consider, for example, FIG. 4 which illustrates an example 400 of a connector for connecting an external communications module to an interactive object in accordance with one or more implementations. In example 400, interactive object 104 is illustrated as a jacket.

As described above, interactive object 104 includes an internal electronics module 204 which include various types of electronics, such as sensing circuitry 210, sensors (e.g., capacitive touch sensors woven into the garment, microphones, or accelerometers), output devices (e.g., LEDs, speakers, or micro-displays), electrical circuitry, and so forth.

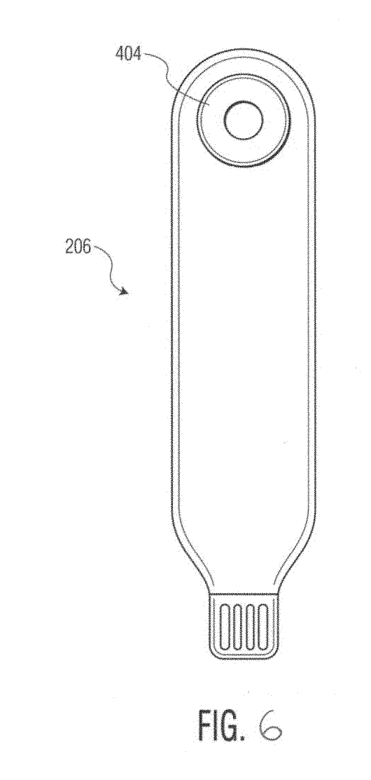

External electronics module 206 which is also shown in FIG. 6 includes various electronics that are configured to connect and/or interface with the electronics of internal electronics module 204. Generally, the electronics contained within external electronics module 206 are different than those contained within internal electronics module 204, and may include electronics such as microprocessor 212, power source 214 (e.g., a battery), network interface 216 (e.g., Bluetooth or WiFi), sensors (e.g., accelerometers, heart rate monitors, or pedometers), output devices (e.g., speakers, LEDs), and so forth.

In this example, external electronics module 206 is implemented as a strap that contains the various electronics. The strap, for example, can be formed from a material such as rubber, nylon, or any other type of fabric. Notably, however, external electronics module 206 may take any type of form. For example, rather than being a strap, external electronics module 206 could resemble a circular or square piece of material (e.g., rubber or nylon).

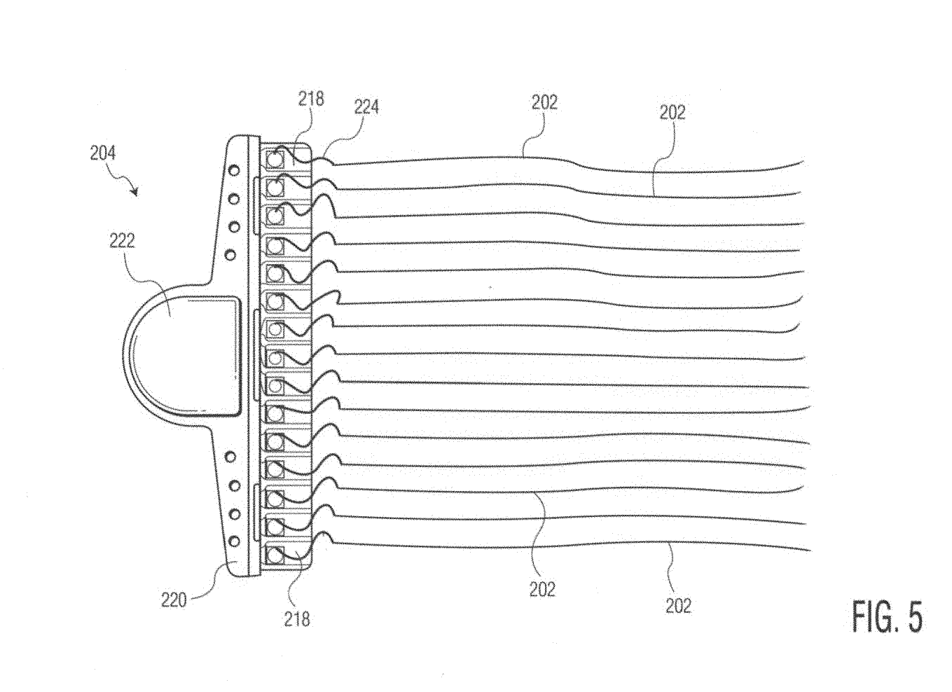

Referring to FIG. 5, the internal electronics module 204 is shown in more detail. The internal electronics module 204, for instance, can be located on the interior of the sleeve as shown in FIG. 4 and attached to connector receptacle 406. In the embodiment illustrated in FIG. 5, the conductive threads 202 are also illustrated. The conductive threads 202 are substantially linear from end to end and form a parallel array.

As shown in FIG. 5, the internal electronics module 204 includes, in one embodiment, a plurality of electrical contact pads 218. The electrical contact pads 218 are spaced sequentially along the width of a flexible substrate 220. The flexible substrate and the electrical contact pads, for instance, may comprise a flexible printed circuit board. The printed circuit board can include a first portion containing the electrical contact pads and a second portion 222 that is in communication with the electrical contact pads. The second portion 222 may comprise a controller or be part of a controller and can include a microprocessor, a network interface, one or more sensors, output devices, and the like.

As described above, the conductive threads 202 are generally in a parallel arrangement. Each conductive thread 202 is connected to a separate and corresponding electrical contact pad 218.

The internal electronics module 204 can include a receptacle 406 that can be used to communicate with the external electronics module 206 as shown in FIG. 4.

For instance, referring to FIGS. 4 and 6, connector 402 includes the connector plug 404 and a connector receptacle 406. In this example, connector plug 404 is positioned on external electronics module 206 and is configured to attach to connector receptacle 406, which is positioned on interactive object 104, to form an electronic connection between external electronics module 206 and interactive object 104. For example, in FIG. 4, connector receptacle 406 is positioned on a sleeve of interactive object 104, which is illustrated as a jacket. In one embodiment, as shown in FIG. 4, the jacket can include a small pocket or opening that can receive the second end of the external electronics module 206.

In various implementations, connector plug 404 may resemble a snap or button, and is configured to connect or attach to connector receptacle 406 via a magnetic and/or mechanical coupling. For example, in some implementations magnets on connector plug 404 and connector receptacle 406 cause a magnetic connection to form between connector plug 404 and connector receptacle 406. Alternately, a mechanical connection between these two components may cause the components to form a mechanical coupling, such as by “snapping” together.

One or more of the electronic modules may comprise an output device configured to provide a haptic response, an audio response, a visual response, or some combination thereof. Examples of haptic responses include vibration, compression or constriction, relaxation, or mixtures thereof. Examples of audio responses include verbal announcements, chimes, tones, or music. Examples of visual responses include displayed text, charts, graphs, light arrays, or glowing colors. Example combinations of the response types might include visual responses synchronized with audio responses; optionally, audio responses may also correspond with haptic responses.

The output device may optionally be integrated into the object 104 or may be embodied by a separate device. For example, the internal electronics module 206 may comprise an output device which includes a liquid crystal or e-ink display within the object 104. In another embodiment, the output device may be separate from the object 104, such as a smartphone 106-2 in communication with the object 104.

Example Computing System

In general, it is to be understood that the interactive object 104 may comprise multiple electronic modules. These modules may also be in communication with a controller. The controller may optionally be integrated into the object or may be a separate device. For example, the controller may be embedded in the interactive garment and accept raw touch-input signal data from the internal electronics module 204 and pass processed data to an external device, such as a smartphone 106-2. In another embodiment, the controller is a smartphone 106-2 external to the garment that receives wireless signals broadcast by the internal and/or external electronic modules that lie within the garment. In such an embodiment, the smartphone 106-2 may optionally return instructions or data responsive to the touch-inputs of the user to an output device within the object 104; alternatively, the smartphone 106-2 may provide instructions or data responsive to the touch-inputs of the user to an external output device (which may include the smartphone itself, in some examples).

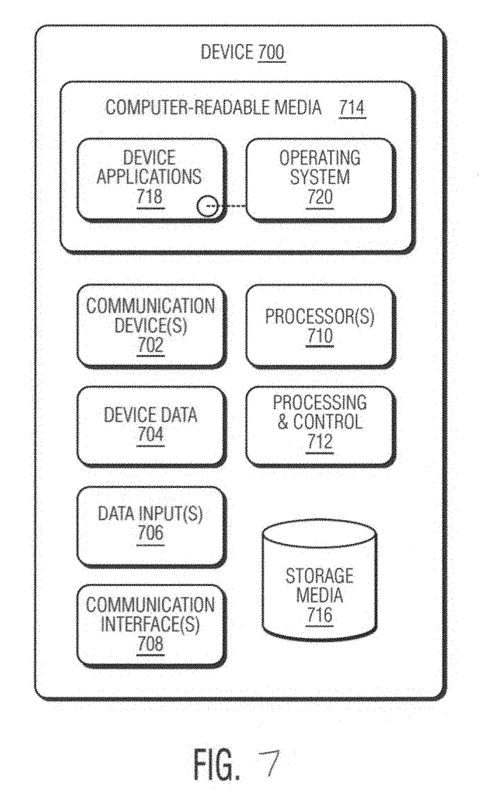

FIG. 7 illustrates various components of an example computing system 700 that can be implemented as any type of client, server, and/or computing device as described with reference to the previous FIGS. 1-6 to implement an interactive object with multiple electronics modules. For example, computing system 700 may correspond to external electronics module 206 and/or embedded in interactive object 104. In embodiments, computing system 700 can be implemented as one or a combination of a wired and/or wireless wearable device, System-on-Chip (SoC), and/or as another type of device or portion thereof. Computing system 700 may also be associated with a user (e.g., a person) and/or an entity that operates the device such that a device describes logical devices that include users, software, firmware, and/or a combination of devices.

Computing system 700 includes communication devices 702 that enable wired and/or wireless communication of device data 704 (e.g., received data, data that is being received, data scheduled for broadcast, data packets of the data, etc.). Device data 704 or other device content can include configuration settings of the device, media content stored on the device, and/or information associated with a user of the device. Media content stored on computing system 700 can include any type of audio, video, and/or image data. Computing system 700 includes one or more data inputs 706 via which any type of data, media content, and/or inputs can be received, such as human utterances, user-selectable inputs (explicit or implicit), messages, music, television media content, recorded video content, and any other type of audio, video, and/or image data received from any content and/or data source.

Computing system 700 also includes communication interfaces 708, which can be implemented as any one or more of a serial and/or parallel interface, a wireless interface, any type of network interface, a modem, and as any other type of communication interface. Communication interfaces 708 provide a connection and/or communication links between computing system 700 and a communication network by which other electronic, computing, and communication devices communicate data with computing system 700.

Computing system 700 includes one or more processors 710 (e.g., any of microprocessors, controllers, and the like), which process various computer-executable instructions to control the operation of computing system 700 and to enable techniques for, or in which can be embodied, interactive textiles. Alternatively or in addition, computing system 700 can be implemented with any one or combination of hardware, firmware, or fixed logic circuitry that is implemented in connection with processing and control circuits which are generally identified at 712. Although not shown, computing system 700 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

Computing system 700 also includes computer-readable media 714, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. A disk storage device may be implemented as any type of magnetic or optical storage device, such as a hard disk drive, a recordable and/or rewriteable compact disc (CD), any type of a digital versatile disc (DVD), and the like. Computing system 700 can also include a mass storage media device 716.

Computer-readable media 714 provides data storage mechanisms to store device data 704, as well as various device applications 718 and any other types of information and/or data related to operational aspects of computing system 700. For example, an operating system 720 can be maintained as a computer application with computer-readable media 714 and executed on processors 710. Device applications 718 may include a device manager, such as any form of a control application, software application, signal-processing and control module, code that is native to a particular device, a hardware abstraction layer for a particular device, and so on. Device applications 718 also include any system components, engines, or managers to implement an interactive object with multiple electronics modules.

### OTHER EMBODIMENTS

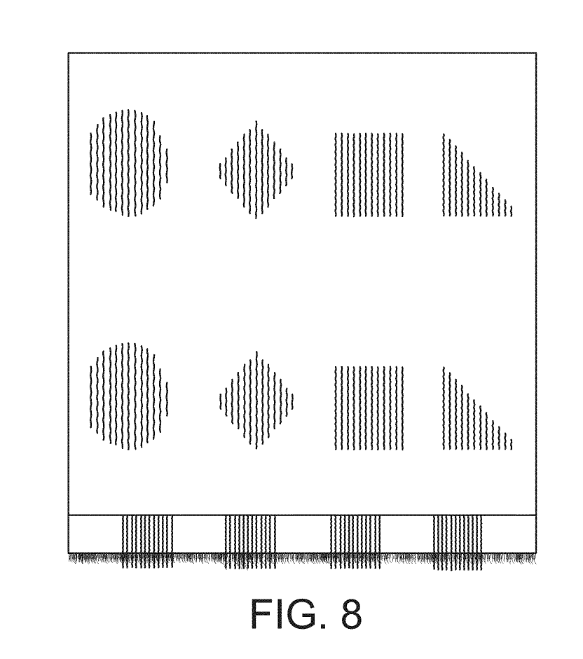

Referring now to FIGS. 8-32, various other embodiments and aspects of the present disclosure are illustrated.

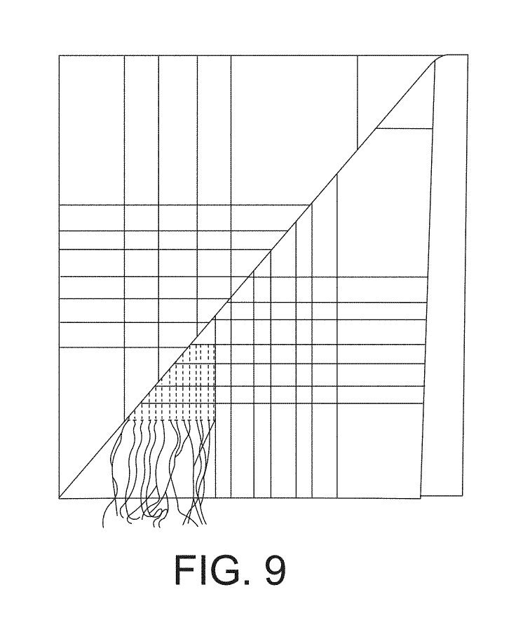

For example, FIGS. 8 and 9 represent embodiments of textiles or fabrics that may be woven into specific patterns in accordance with the present disclosure. In FIG. 8, the conductive yarns form geometrical shapes within a fabric. In FIG. 9, the conductive yarns are incorporated into the fabric so as to be invisible.

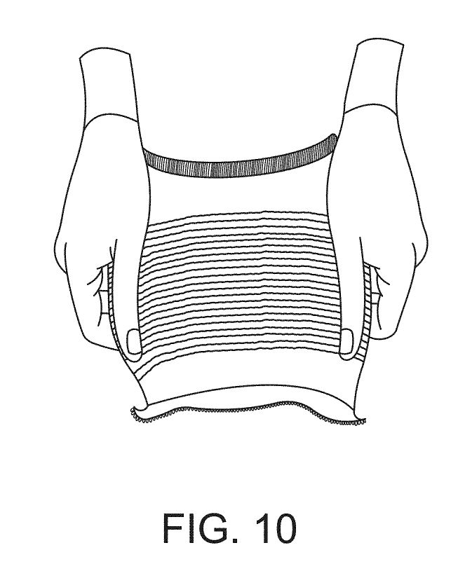

The conductive yarns of the present disclosure can also be incorporated into fabrics or textiles having elasticity. For instance, FIG. 10 illustrates a knitted fabric incorporating conductive yarns. The knitted fabric is stretchable.

The above-mentioned fabrics can be conducive to a number of useful embodiments. For example, a blanket can be provided with a touch sensor and associated electronics modules to control a television or other entertainment device. In another embodiment, a mat can be augmented with conductive yarns and associated electronics modules to facilitate games or exercise activities on the mat.

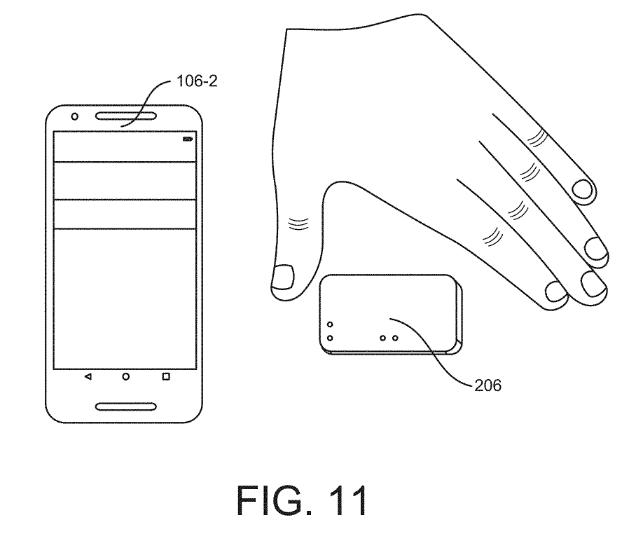

Referring to FIG. 11, an electronic module 206 made in accordance with the present disclosure is illustrated in proximity to a computing device 106. The electronic module 206 is designed to be directly or indirectly connected to one or more conductive threads. The electronic module 206 includes an integrated circuit chip for communication with the computing device 106 through a suitable communication device or system, such as Bluetooth. In the embodiment illustrated in FIG. 11, the person's hand is placed in close proximity to the module 206 which causes a display to occur on the computing device 106.

In one embodiment, the conductive yarns in conjunction with one or more electronic modules can control flexible haptics without the need for a motor.

For example, the conductive yarns may permit the user to control a number of “smart material” actuators; piezoelectric materials, electroactive polymers, and dielectric elastomers all exemplify materials which can provide a haptic response to a touch-input instruction from a user without the need for a motor. For example, electrically activated materials (e.g. those listed above) can be used to induce torsional and/or linear motion responsive to an electrical signal. In one embodiment, multiple segments composed of a piezoelectric composite may be linked together in a ring shape; application of electrical potential across the electrodes of each segment can increase or decrease the diameter of the ring. In another example, electroactive polymers can be used to change or adapt the texture of the surface of an interactive object 104 responsive to an applied electrical field.

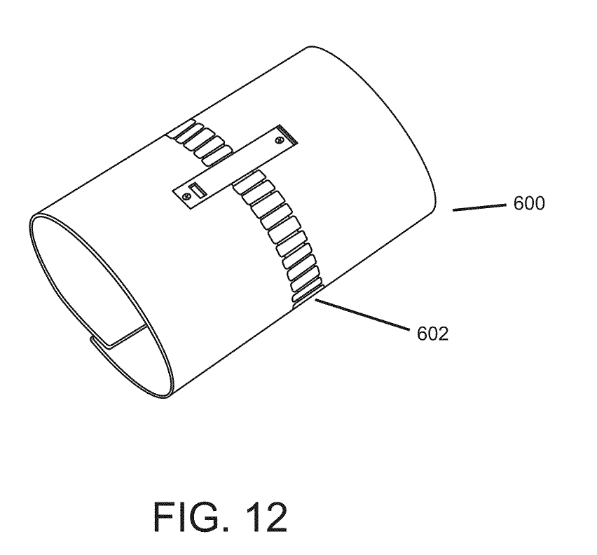

For example, one embodiment of a flexible haptic device is shown in FIG. 12. The example device shown is a haptic cuff 600; the haptic cuff 600 can be controlled by a controller integrated into one or more electronic modules and attached to the conductive yarns. The haptic cuff 600 includes a contraction band 602 that can be designed to expand or contract an area of a garment. In one example, the haptic cuff 600 can be placed within the wrist cuff or ankle cuffs of a shirt or a pair of pants, respectively. Responsive to a touch-input from the wearer, the cuffs could expand or contract to suit the wearer's comfort needs. In another embodiment, a similar contraction band 602 could be placed around a user's waistband; for example, such a configuration would permit the style of a dress or shirt to be adjusted extemporaneously without requiring a private changing room or any awkward manipulations of the garment.

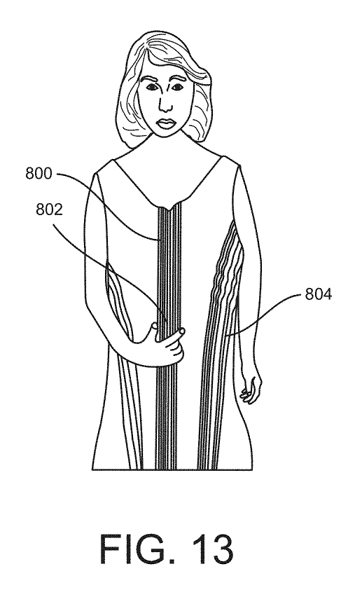

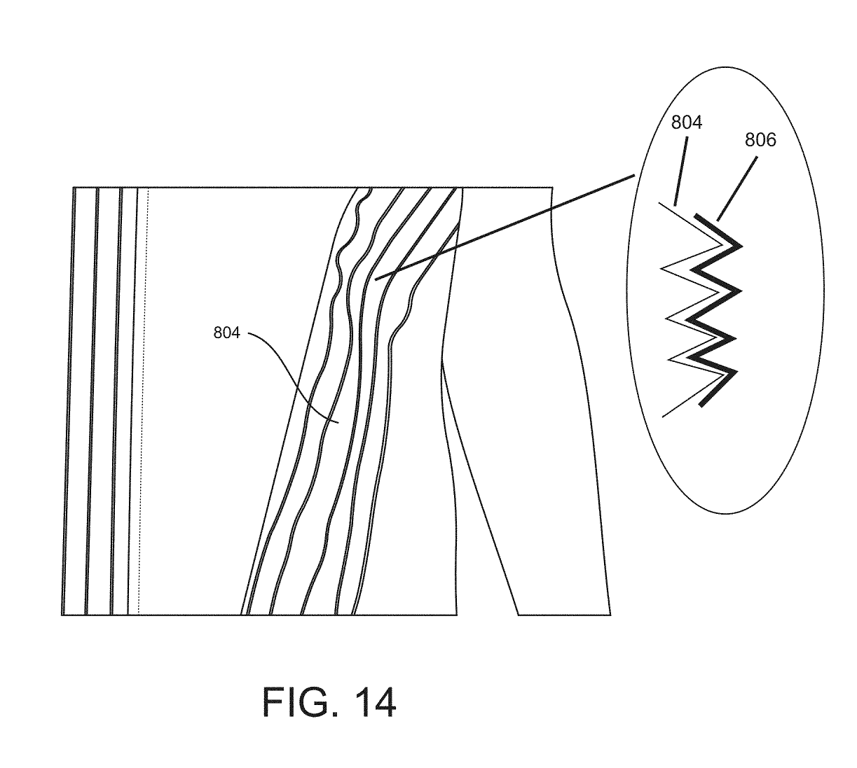

In another example, referring to FIGS. 13 and 14, one embodiment of a garment is illustrated that includes areas that can be expanded and retracted in response to interaction with a capacitive touch sensor. As shown in FIG. 13, the garment 800 includes a capacitive touch sensor 802. The user contacts the touch sensor with a particular motion or gesture. The input to the capacitive touch sensor 802 is communicated to an electronic module that then controls interactive features of the garment 800.

For instance, as shown in FIG. 14, the garment can include a plurality of pleats 804 that can expand or contract based upon the user input. As previously discussed, the pleats may be actuated by any number of materials which eliminate the need for a motor. For example, the fabric pleats 804 may be reinforced in certain portions by piezoelectric composite pleat actuators 806, where the fold of each pleat is formed at the intersection of two piezoelectric composite pleat actuators 806 of opposite electrical polarity. In such a configuration, the applied electrical field across the pleat actuators 806 will induce an accordion effect, collapsing or expanding the pleats 804.

A separate computing device, such as a smartphone, can monitor change in the garment and inform a user how much the garment has expanded or contracted. Based on the readings on the smartphone, the user can decide whether to further adjust the garment 800 using the capacitive touch sensor 802.

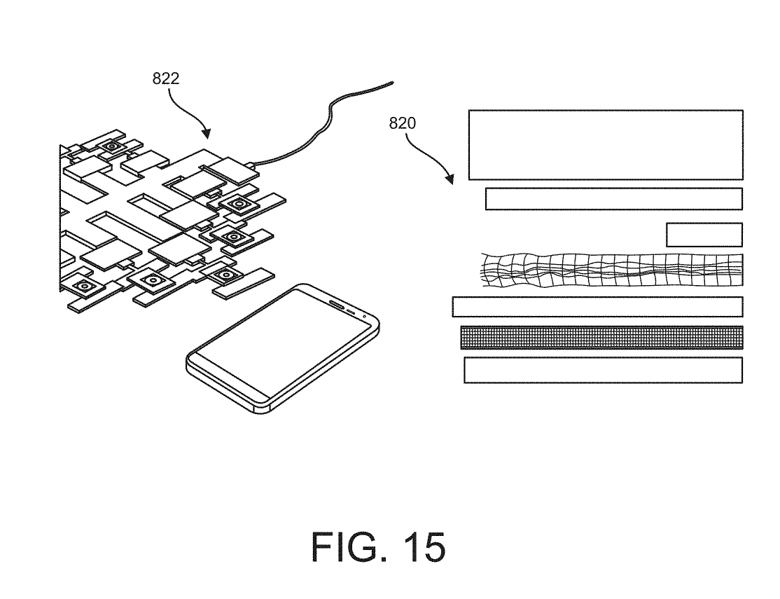

In one embodiment, the conductive yarns can be incorporated into ribbons for producing different circuit configurations within a garment. For example, referring to FIG. 15, a plurality of different ribbons 820 are shown that may be incorporated into interactive textiles in accordance with the present disclosure. Each of the ribbons includes conductive yarns spaced in a parallel and/or intersecting relationship. The ribbons 820 can be used to produce a wearable network 822 as shown in FIG. 15. The wearable network can be connected to various printed circuit boards, LED devices, audio devices, and the like. The network 822 can be controlled or monitored by a smartphone or notebook computer as shown in FIG. 15.

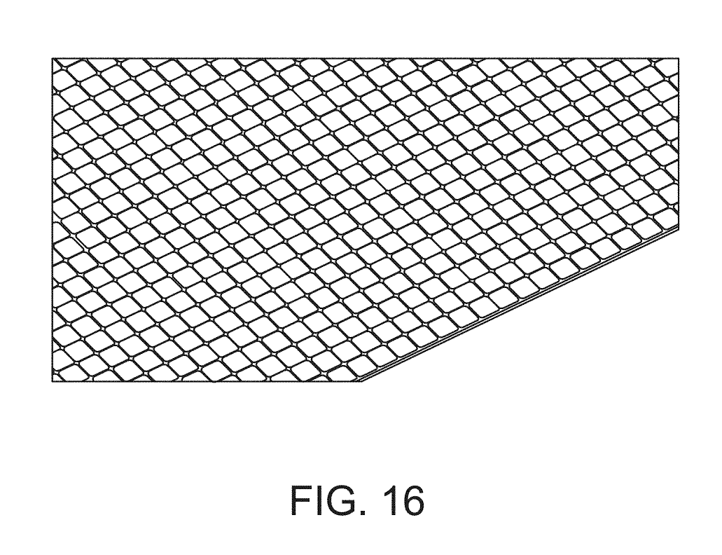

FIG. 16 illustrates another textile that can be made in accordance with the present disclosure. The textile illustrated in FIG. 16 includes a pattern of geometric shapes. The pattern includes large square shapes and smaller square shapes. In accordance with the present disclosure, light-emitting diodes can be embedded within the large shapes and within the small shapes in any suitable desired pattern. The conductive yarns can be connected to the light-emitting diodes for causing the light-emitting diodes to light up in a particular decorative pattern.

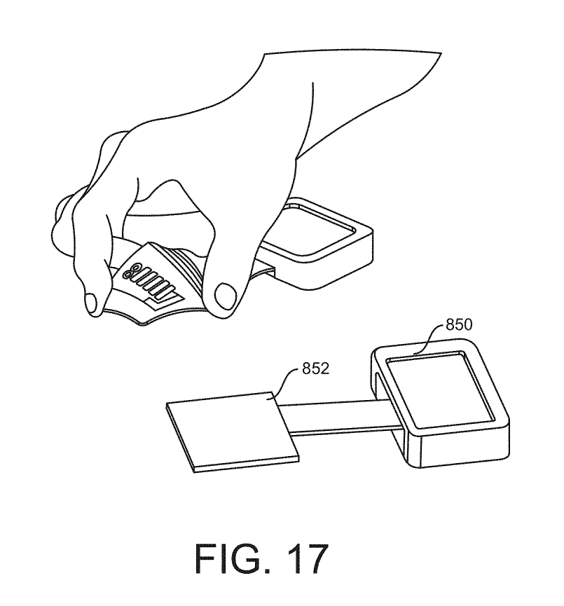

Referring to FIG. 17, another embodiment of an electronics module 850 is illustrated. The electronics module 850 is designed to connect to a plurality of conductive yarns or threads within an interactive object or textile. As shown, the electronics module 850 includes or is attached to a flexible printed circuit board 852. The flexible printed circuit board 852, in one embodiment, may comprise a plurality of light-emitting diodes, such as organic light-emitting diodes. The light-emitting diode as shown in FIG. 17 is exceptionally flexible. Thus, the light-emitting diode can be easily integrated into textiles. The light-emitting diode may include a display for displaying information or can be included for decorative purposes.

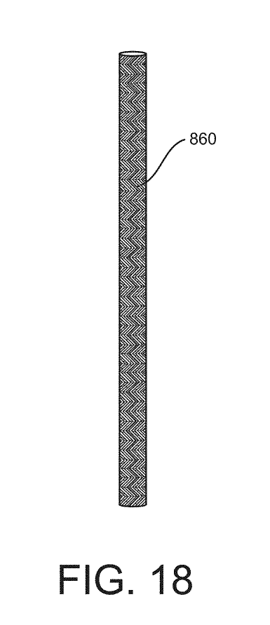

In one embodiment, the interactive object or textile may include a touch cord 860 as shown in FIG. 18. The touch cord can be comprised of one or more conductive yarns and can be connected to an electronics module. The touch cord 860 can operate as a touch sensor; for example, the touch cord 860 may operate as a capacitive touch sensor 802. For example, a user can apply different forces to the touch cord 860 which are then interpreted by a controller in order to carry out a desired operation. For example, the hands of a user can slide along the touch cord 860, can twist the touch cord 860, can pull and stretch the touch cord 860, or can squeeze the touch cord 860 in a particular manner that provides touch-input instructions to an electronics module for controlling an electronic device within the interactive object.

The touch cord 860 may be combined with other input-sensing technologies. For example, in another embodiment, the touch cord 860 also comprises a sealed, gas-filled cavity extending the length of the cord. A pressure sensor connected along any portion of the touch cord may record both the occurrence of a pinch or squeeze and also the intensity. For example, such a system may work alongside a capacitive touch cord 860 to distinguish “soft swipe” instructions and “hard swipe” instructions and assign varied responses to each.

The touch cord 860 may be combined with other electronic cords. For example, the touch cord 860 may further comprise an audio cable, allowing a user to control the playback in connected devices. For example, the touch cord audio cable may transmit audio signals from a smartphone to a stereo and provide an interactive control interface for music playback. In another example, the touch cord audio cable may transmit audio signals from a laptop to a pair of headphones and provide an interactive control interface for headphones that might otherwise lack such a feature.

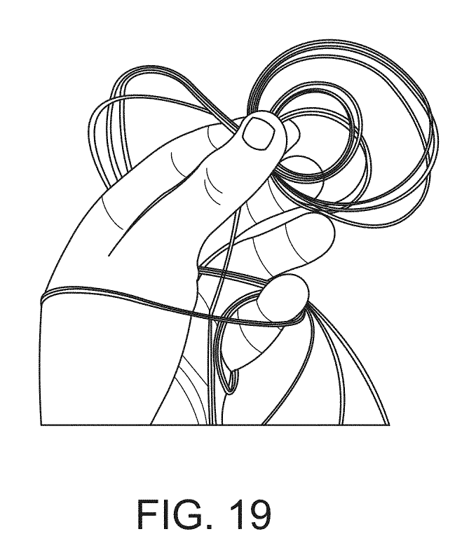

FIG. 19 shows various flexible lighting solutions that may be incorporated into the interactive object.

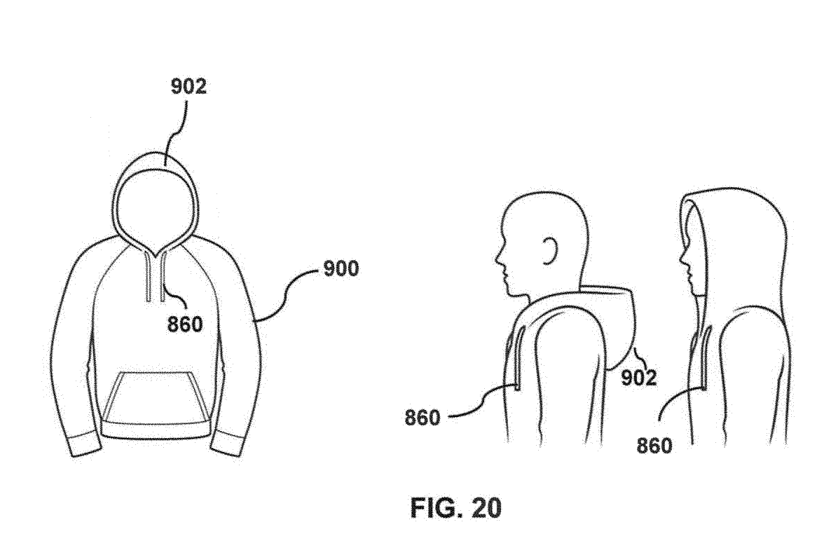

One embodiment of an interactive object made in accordance with the present disclosure is illustrated in FIG. 20. In FIG. 20, the interactive object comprises a garment, such as a “hoodie” 900. The garment 900 includes a hood 902. In accordance with the present disclosure, the garment 900 includes one or more touch cords 860 that comprise drawstrings along the hood 902.

The touch cord 860 is placed in communication with an electronics module. For example, the touch-input instructions gathered by the touch cord 860 may be used to control the electronics module directly, or in another example, the electronics module may relay the touch-input instructions to a computing device 106, such as a smartphone, for execution of the touch-input instructions.

The electronics module controls, in one embodiment, lightweight speakers mounted within the hood. The electronics module also is capable of connecting the speakers to a music playing device, such as a smartphone. In this manner, a user can provide touch input to the touch cord 860 for turning on and off music, adjusting volume, selecting a song or playlist, or the like. As shown in FIG. 20, the hood 902 can be placed over the wearer's head so that the wearer can listen to music or other audio individually. Alternatively, the hood 902 can be draped around the neck of the wearer so that others in the area can listen to the audio being supplied by the speakers.

The electronics module may also provide active noise cancellation in some embodiments. For example, the hood 902 may include microphones to detect ambient sound or noise outside the hood 902. The electronics module may then provide a noise-cancelling signal to the speakers in the hood 902.

The electronics module controls, in another embodiment, a computing device 106 through a network interface 216. For example, one embodiment may use touch-input instructions from the touch cord 860 to control the playback of music on a home audio system connected to the electronics module by a WiFi signal. In another example, the touch-input instructions may be relayed via Bluetooth from the electronics module to a smartphone 106-2 or smart watch 106-9 to control the playback of music on a wireless headset worn by the user (e.g. Bluetooth-connected earbuds). In another embodiment, microphones within the hood 902 may facilitate hands-free phone conversations in conjunction with a connected smartphone.

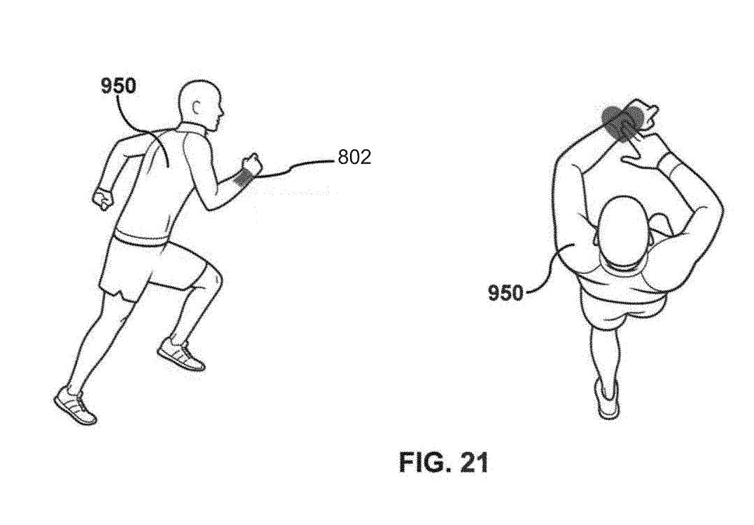

Referring to FIG. 21, another interactive garment 104 made in accordance with the present disclosure is shown. In this embodiment, the garment 104 can comprise an athletic shirt 950. The athletic shirt 950 can include a capacitive touch sensor 802 located on the sleeve. The capacitive touch sensor 802 can comprise an array of conductive yarns in conjunction with one or more electronics modules. The electronics modules, for example, may include sensors to monitor athletic performance or the health of the wearer.

For example, one or more of the electronics modules may comprise a GPS sensor, enabling the electronics module to monitor the location, trajectory, and path/or history of the wearer of the shirt 950. Optionally, the electronics modules may communicate with the user's smartphone or smartwatch to obtain location, trajectory, or path data. In another embodiment, one or more of the electronic modules may comprise electrocardiogram (ECG) sensors. In another embodiment, the ECG sensors include one or more conductive threads 202. The conductive threads 202 distributed throughout the shirt 950 permit subtle and comfortable placement of ECG conductors around the chest and on the arms of the wearer. The information collected by the electronics modules from the ECG sensors may be available to an output device alone or may also be sent to a computing device for convenient record keeping.

In addition, the capacitive touch sensor 802 can, in one embodiment, incorporate a light-emitting diode (e.g. OLED) for providing a visual display. The visual display can be used to check speed, distance, and pace during use of the athletic shirt 950. For example, the visual display may be a glowing color, with various colors or color intensities mapped to various positions, locations, or postures within a predetermined routine; a quick glance may inform the wearer of progress. In another embodiment, a glowing display may provide a quick indication of the wearer's heart rate. In another embodiment, a more detailed visual display may provide a map overlaid with a runner's route. The map data may be served by an electronics module within the shirt 950 or may be collected, for example, from a smartphone or smartwatch in communication with the electronics module(s) within the shirt 950. As also shown in FIG. 21, touch-input instructions on the capacitive touch sensor can also be used to control other functions such as monitoring heart rate.

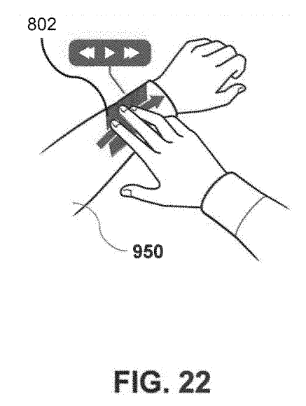

Referring to FIG. 22, the capacitive touch sensor 802 is shown in the form of parallel stripes in one embodiment. It should be understood, however, that the capacitive touch sensor can be made to have any desired appearance. As shown in FIG. 22, various different hand motions that contact the capacitive touch sensor 802 can be used to indicate different instructions. For instance, the manner in which the capacitive touch sensor 802 is contacted can be used to control music, music volume, a workout timer, an exercise repetition (“reps”) counter, or any of the functions described with respect to FIG. 21. Example hand motions include tapping motions, swiping or brushing motions in any direction, compressive motions (e.g. squeezing a wrist cuff), or combinations thereof.

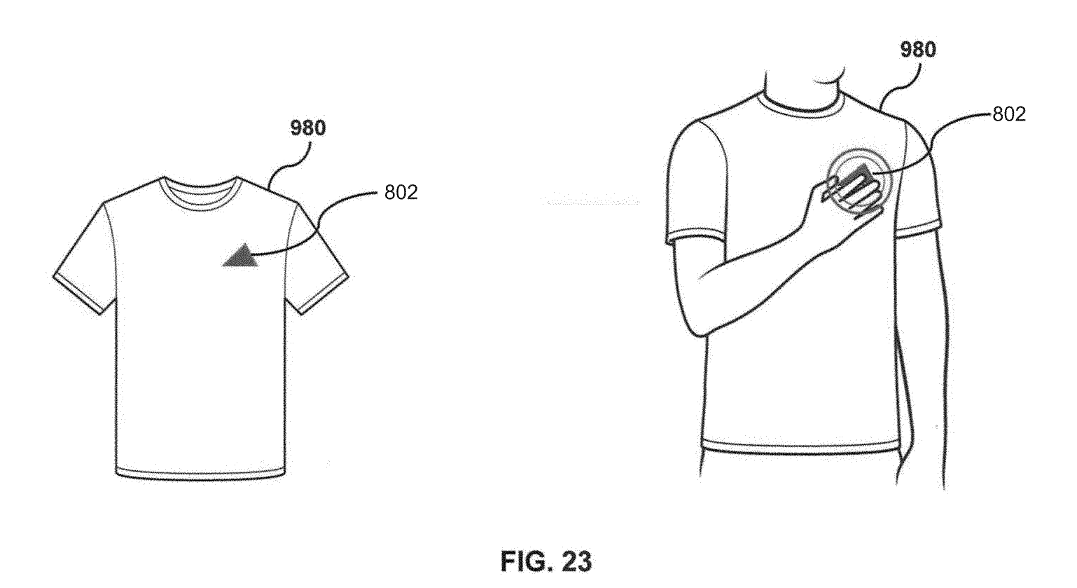

Yet another embodiment of an interactive object or garment 104 is illustrated in FIG. 23. The garment illustrated in FIG. 23 comprises a shirt 980 that includes a capacitive touch sensor 802. In this embodiment, the capacitive touch sensor is a design or icon located on the front of the shirt. Touching or tapping the capacitive touch sensor 802 in a particular manner provides instructions to one or more electronics modules for controlling one or more functions. For instance, the capacitive touch sensor 802 may be used to control music on a smartphone or other music playing device; in addition to controlling playback, tapping the icon to a particular rhythm or beat, for example, can guide a connected electronic module or smartphone in selecting a particular song which matches the prescribed rhythm or beat. In addition, the capacitive touch sensor 802 can include one or more light-emitting diodes. The light-emitting diodes may light up while music is playing. In one embodiment, for instance, the light-emitting diodes may be configured to illuminate in a pattern that corresponds with the beat of the music.

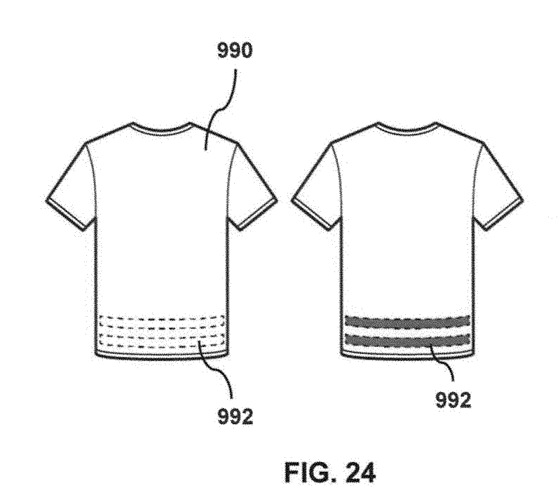

Referring to FIG. 24, still another embodiment of an interactive object or garment 104 is shown. In the embodiment illustrated in FIG. 24, the shirt 990 includes at least one safety illumination device 992, such as a plurality of stripes. The shirt 990 further includes a capacitive touch sensor. The capacitive touch sensor can be connected to an electronics module which controls the safety illumination device 992. Touch input to the capacitive touch sensor can cause the safety illumination device 992 to light up so that the wearer of the shirt can be seen at night.

In one embodiment, the safety illumination device or stripes 992 can be connected to the electronics module which monitors movement of the wearer. The stripes 992 can change color depending upon movement. For instance, the stripes can turn red if the wearer stops suddenly.

The safety illumination device or stripes 992 can also be controlled by the wearer so as to illuminate in a particular manner depending upon input received on the capacitive touch sensor. For instance, the user can cause the stripes 992 to flash or to otherwise indicate that the wearer is turning a particular direction.

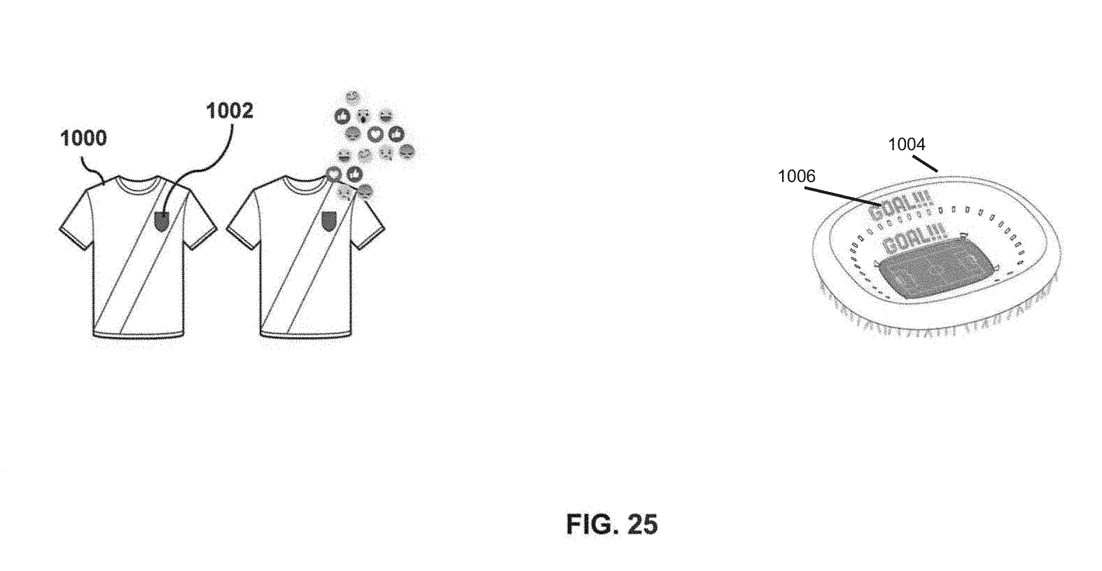

Referring to FIG. 25, still another embodiment of an interactive object or garment is shown. The shirt 1000 can include a capacitive touch sensor 802 in conjunction with one or more electronic modules as described in detail above. In the embodiment illustrated in FIG. 25, the shirt 1000 is designed to be a team jersey or team apparel. The shirt 1000 includes an interactive icon 1002 into which the capacitive touch sensor 802 is integrated. The icon 1002 can also include one or more light-emitting diodes that can illuminate the icon at desired times.

As shown in FIG. 25, in one embodiment, the shirt 1000 can be worn during a sporting event. A user can then use the capacitive touch sensor 1002 in order to control various functions on the garment or various functions related to a computing device such as a smartphone. For instance, in one embodiment, the capacitive touch sensor 1002 can be configured to receive user input for automatically sharing messages on social networks.

In one embodiment, the interactive garment 1000 can also be configured to recognize and locate similar garments worn by other fans. For instance, when two similar garments (i.e. that have the same team colors) come in to close proximity, the icon 1002 can automatically light up. The icon 1002 with the capacitive touch sensor 802 can also be used to share one's location on social networks.

As can be appreciated, the shirt 1000 can include multiple light-emitting diodes so that the entire garment or large portions of the garment can illuminate and/or change colors. In one embodiment, for instance, the garments can be collectively controlled instantaneously. In this manner, the garments can display a synchronized choreography. For example, spectators on one side of the stadium 1004 may participate in the choreographed graphic 1006 to display a cheer or celebrate a goal; each participating spectator's shirt 1000 displays a subsection of the larger choreographed graphic 1006.

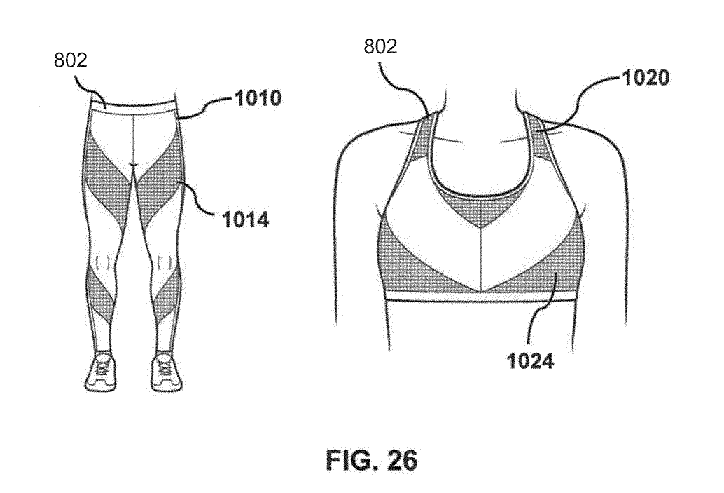

As described above with respect to FIGS. 12-14, garments made according to the present disclosure can include various different haptics devices that can provide compression or relaxation. The garment can include a capacitive touch sensor for receiving user input for controlling the haptics devices. For example, referring to FIG. 26, various interactive garments 1010 and 1020 are illustrated. Interactive garment 1010, for instance, comprises compression pants while interactive garment 1020 illustrates a compression bra. The compression pants 1010, for instance, can include a capacitive touch sensor 802 that is in communication with compression panels 1014. The capacitive touch sensor 802 in conjunction with one or more electronics modules can be used to increase or lower the level of compression placed on the legs of the wearer by the compression panels 1014.

Similarly, the compression bra 1020 can include a capacitive touch sensor 802 that is in communication with a plurality of compression panels 1024. The compression panels 1024 can be controlled by the capacitive touch sensor 802 in order to cycle through different levels of compression.

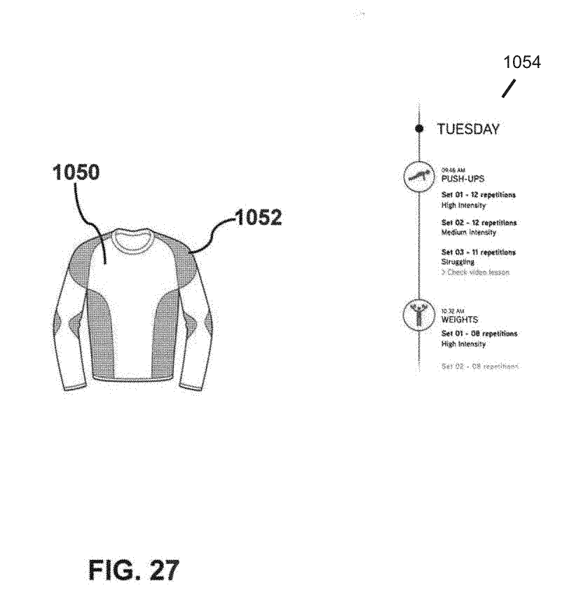

Referring to FIG. 27, still another embodiment of an interactive object or garment 104 is shown. The shirt 1050 comprises an exercise shirt that includes a plurality of sensor pads 1052.

The sensor pads 1052 may include capacitive touch sensors as previously described herein. Additionally or alternatively, the sensor pads 1052 may include one or more of accelerometers, inertial measurement units, gyroscopes, piezoelectric sensors, altimeters, optical sensors, temperature sensors, or health measurement sensors, such as ECG sensors, electroencephalography (EEG) sensors, or respiration monitors. In addition to the sensors, each sensor pad 1052 may comprise an internal clock to enable concurrent or reconstructive synchronization of all measurements collected by a plurality of sensor pads 1052. In some embodiments, each sensor pad 1052 may include connectivity options, such as wired or wireless communication devices which permit operation in conjunction with a computing device, e.g. a smartphone.

The sensor pads 1052 can, in one embodiment, be controlled by a capacitive touch sensor located at any suitable location on the garment. The sensor pads 1052 can be comprised of an array of conductive yarns that sense the movement of the wearer. For instance, the sensor pads 1052 can be positioned around joints that sense and record movement of the individual wearing the garment. In this manner, the shirt 1050 can automatically recognize different types of exercise and provide statistics. For instance, the garment can count repetitions and provide performance statistics. This information can be displayed on a display pad incorporated into the garment or can be displayed on a computing device, such as a smartphone. The garment can communicate with a computing device or smartphone through the use of one or more electronic modules. The information collected by the garment or a connected computing device may be displayed in a manner such as shown in the example exercise log 1054.

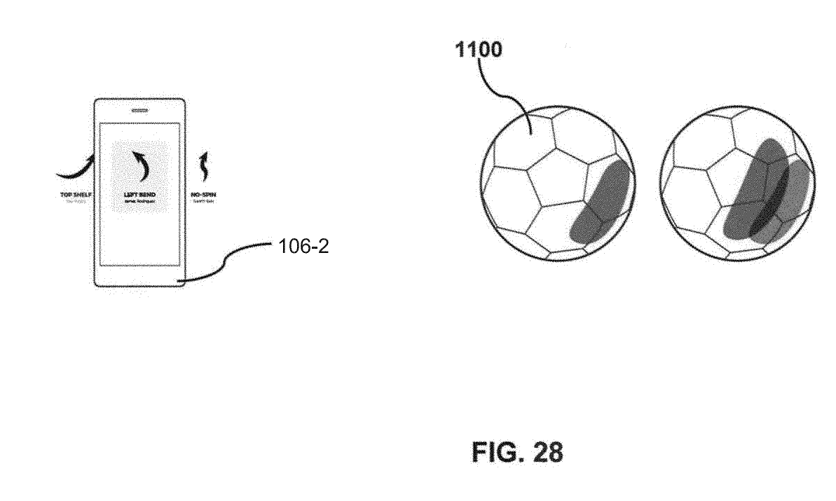

In addition to garments, the system of the present disclosure can be incorporated into other various articles and products. For instance, in one embodiment, the system of the present disclosure can be incorporated into various sports equipment. For example, referring to FIG. 28, an interactive object that, in this embodiment, comprises a soccer ball 1100 is shown. The ball 1100, for instance, can include a capacitive touch sensor. For example, the ball 1100 can be made with conductive threads. In one embodiment, the conductive threads can form a capacitive touch sensor that covers substantially the entire surface area of the ball. The ball can include one or more electronic modules for communicating with a computing device, such as a smartphone 106-2. The capacitive touch sensor incorporated into the ball 1100 can be used to determine where the ball has been kicked or otherwise touched for providing information to the user.

In one embodiment, as shown in FIG. 28, the smartphone can be used to learn different skills such as placing spin on the ball 1100 when kicked. The smartphone 106-2 can communicate with the ball 1100 and provide a user a location on the ball where the ball should be contacted for a particular result to occur. In one embodiment, the ball can be configured to display a location to the user where the ball should be contacted and then compare the displayed location with the location where the ball was actually contacted. The difference in contact points can then be displayed on the smartphone 106-2 for improving the skills of the user. In one embodiment, the ball 1100 may operate in communication with an interactive shoe or cleat constructed according to the present disclosure; in such an embodiment, the training aid graphics may be displayed on both the ball 1100 and the cleats.

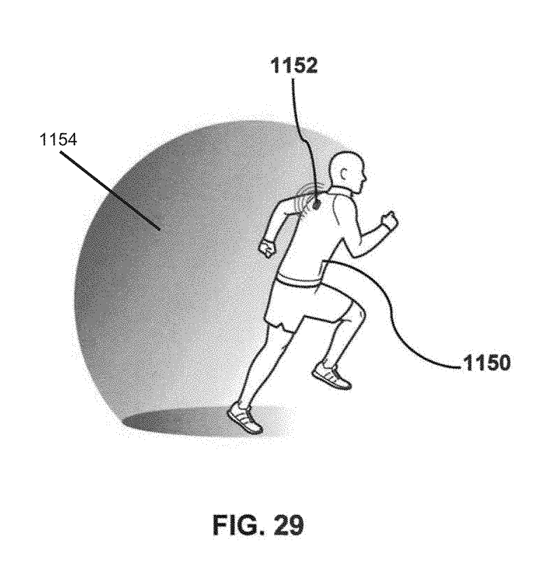

Referring to FIG. 29, still another embodiment of an interactive object or garment 104 is shown. The interactive garment 104 comprises exercise clothing or an exercise shirt 1150 and may incorporate any of the features described above with respect to the garments illustrated in FIGS. 21-27. In FIG. 29, the shirt 1150 further includes a vibration device 1152. The vibration device 1152 can be placed in communication with a type of radar that senses other objects in the vicinity 1154 of the wearer. For instance, a radar device can be incorporated into the garment that senses when other players, vehicles, or other objects come within a specified vicinity 1154 of the wearer. Once an object is sensed, the vibration device 1152 can vibrate alerting the wearer to the presence of the object. In this manner, a person wearing the garment can stay focused on an athletic task without having to worry about other objects approaching from behind.

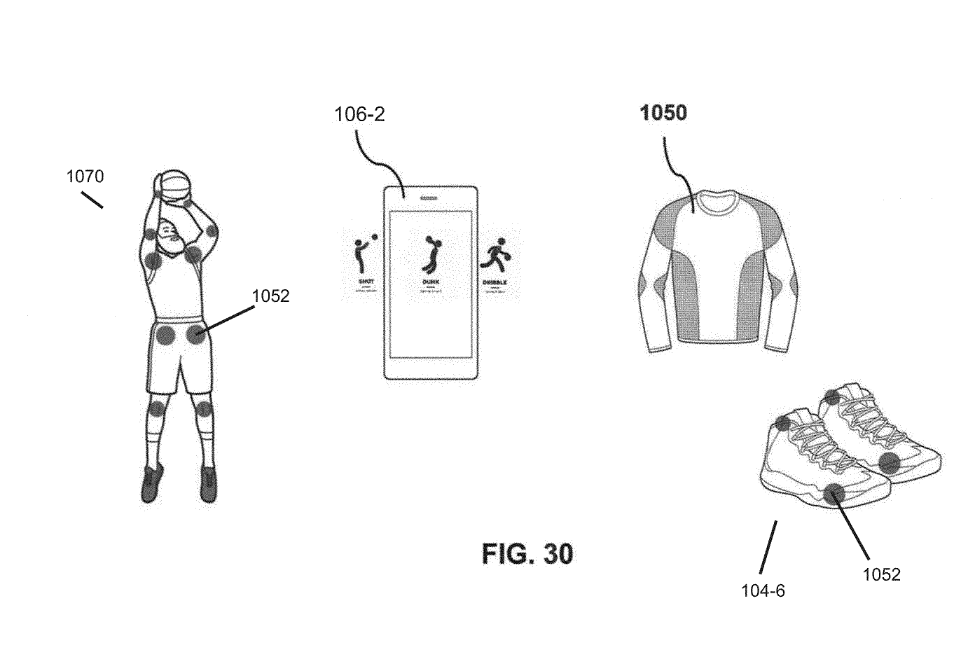

Referring to FIG. 30, the interactive shirt 1050 as shown in FIG. 27 is illustrated. As shown in FIG. 30, the garment 1050 can, in one embodiment, be used to compare the performance of a wearer with the performance of another individual, such as a professional athlete.

For instance, a professional athlete 1070 can wear the shirt 1050 during athletic activity. In some embodiments, the athlete 1070 may wear athletic shorts, kneepads, elbow pads, and wrist bands which also contain sensor pads 1052. The garments can sense movement of the athlete 1070 with the sensor pads 1052 and record the movements. This information can be recorded and transmitted to a smartphone 106-2. In the embodiment illustrated in FIG. 30, for instance, the movements of a professional basketball player 1070 are recorded while the athlete 1070 is playing basketball.

Another user can then wear similar garments during a sporting activity such as when playing basketball. The garments can record performance and compare the data collected during the performance of the user to data collected during a reference performance of a chosen player, such as a professional basketball player. As shown in FIG. 30, the system incorporated into the shirt 1050 can also be incorporated into a pair of shoes 104-6. In this manner, the user may wear the shirt 1050 and/or shoes 104-6 and can imitate and learn from a professional athlete for improving one's skill.

It is to be understood that any performance may be monitored and programmed into a reference library. For example, a library of reference performances may be collected by monitoring fitness trainers as disclosed above. In another example, professional gymnasts, runners, swimmers, or other athletes may be similarly monitored. Accordingly, a user may compare data collected by the user's own garment(s) with any number of reference performances pre-programmed into a library spanning many disciplines. In addition to professional or other “well-known” athletes, users may compare performance among peer groups. Similarly, other embodiments may employ such a platform to facilitate regional or organizational competitions.

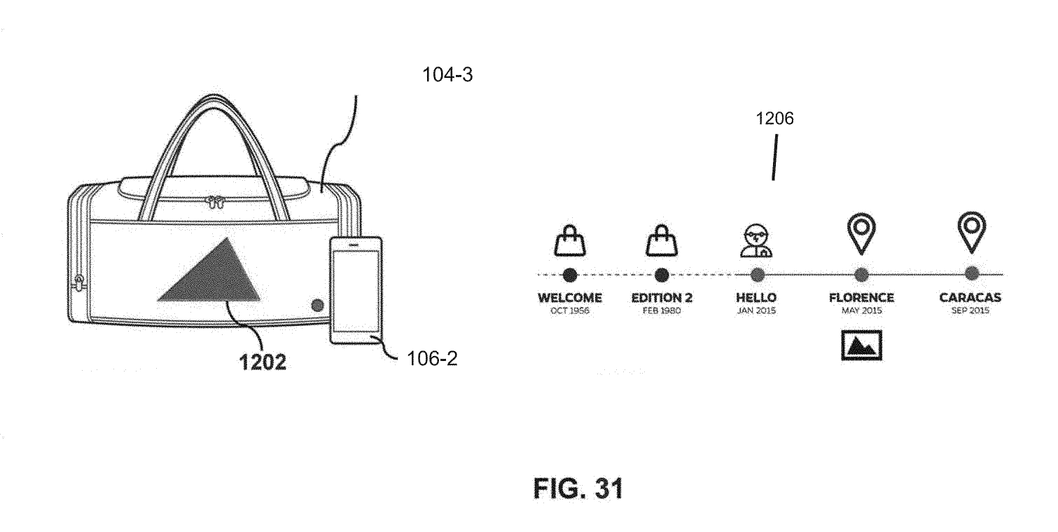

In addition to garments, the system of the present disclosure can also be incorporated into various other articles, such as a handbag 104-3 as shown in FIG. 31. The handbag 104-3 can include a smart tag 1202 which includes conductive yarns. The conductive yarns can be attached to one or more electronic modules for communicating with a computing device, such as a smartphone 106-2. The smart tag 1202 can track the location of the handbag 104-3 and track other activities. The conductive yarns, used to form a touch sensor 802, may facilitate activation or deactivation of the tracking feature, or may control other features, such as those previously disclosed herein. As shown in FIG. 31, this information can be collected over time and can be associated with various photographs taken during travels, as shown in the example timeline 1206. In this manner, the handbag 104-3 can produce a history of travel of the handbag 104-3 on a computing device or smartphone 106-2. It is to be understood that a garment, garment accessory, or garment container of any variety can be modified with such a smart tag to generate a similar history of travel.

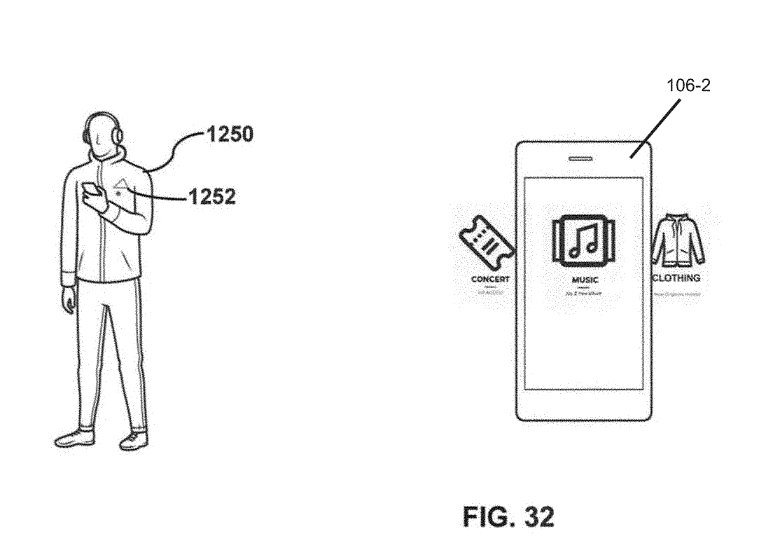

Referring to FIG. 32, still another embodiment of an interactive object or garment 104 made in accordance with the present disclosure is shown. The jacket 1250 includes a smart tag 1252 constructed from one or more conductive yarns in conjunction with one or more electronic modules and/or light-emitting devices. As shown in FIG. 32, in one embodiment, the jacket 1250 can be associated with a corporation that provides exclusive opportunities to the wearer such as initial access to music, clothing lines, concert tickets, and the like.

### CONCLUSION

Although embodiments of techniques using, and objects including, an interactive object with multiple electronics modules has been described in language specific to features and/or methods, it is to be understood that the subject of the appended claims is not necessarily limited to the specific features or methods described. Rather, the specific features and methods are disclosed as example implementations of an interactive object with multiple electronics modules.

What is claimed is:

1. A user-interactive garment, garment accessory, or garment container comprising:
a touch cord comprising a touch sensor, the touch cord being configured to receive touch input from a user;
at least one electronics module associated with the user-interactive garment, garment accessory, or garment container, the electronics module being configured to receive user-inputted actions from the touch cord; and
at least one output device in communication with the electronics module, the at least one output device, based on input received from the electronics module, being configured to provide an output function, the output function comprising one or more of the following:
(1) a haptic response in the user-interactive garment, garment accessory, or garment container;
(2) an audio response; or
(3) a visual response in the output device, the user-interactive garment, the garment accessory, or the garment container.

2. A user-interactive garment, garment accessory, or garment container as defined in claim 1, wherein the touch cord comprises a capacitive touch sensor.

3. A user-interactive garment, garment accessory, or garment container as defined in claim 1, wherein the touch cord comprises a plurality of conductive yarns.

4. A user-interactive garment, garment accessory, or garment container as defined in claim 1, further comprising a controller configured to receive information from the electronics module based upon touch input received from the touch cord.

5. A user-interactive garment, garment accessory, or garment container as defined in claim 1, wherein the user-interactive garment, garment accessory, or garment container comprises the garment.

6. The garment as defined in claim 5, wherein the touch cord comprises a drawstring.

7. The garment as defined in claim 6, wherein the garment comprises a hooded garment including a hood defining an opening, the drawstring extending from the hood.

8. The garment as defined in claim 7, wherein the at least one output device comprises at least one speaker contained within the hooded garment.

9. The garment as defined in claim 8, wherein touch input from a user on the drawstring controls the at least one speaker for providing the audio response.

10. The garment as defined in claim 6, further comprising a controller configured to receive information from the electronics module based upon touch input received from the touch cord.

11. The garment as defined in claim 10, further comprising a network interface, and wherein touch input received by the drawstring is communicated over a wireless network to the controller via the network interface.

12. The garment as defined in claim 10, wherein the controller comprises a mobile device.

13. The garment as defined in claim 10, wherein the at least one drawstring comprises one or more conductive threads that form a capacitive touch sensor, the capacitive touch sensor being in communication with the electronics module, the garment being configured to discern different commands based on different touch types with which the user touches the capacitive touch sensor.

14. The garment as defined in claim 10, wherein the output function is the audio response, and wherein the audio response comprises playing music.

15. A user-interactive garment, garment accessory, or garment container as defined in claim 1, wherein the at least one output device comprises a display device for producing the visual response, the visual response comprising information displayed on the display device.

16. A garment as defined in claim 7, further comprising a controller configured to receive information from the electronics module based upon touch input received from the touch cord.

17. The garment as defined in claim 16, wherein the controller comprises a mobile phone.

18. The garment as defined in claim 17, wherein touch input received by the drawstring is configured to control the mobile phone for answering incoming calls.

19. The garment as defined in claim 17, wherein touch input received by the drawstring is configured to control playing a music library stored on the mobile phone.

20. The garment as defined in claim 9, wherein the electronics module is configured to provide a noise-canceling signal to the at least one speaker contained in the garment.
